(19) United States
(12) Patent Application Publication
(10) Pub. No.: US 2013/0189382 A1
(43) Pub. Date: Jul. 25, 2013

(54) Use of fucoxanthin in the preparation of product for improving memory and having neuroprotective effect associated with neurodegenerative disorder

(71) Applicants: Yanmei Li, Beijing (CN); Liang Li, Beijing (CN)

(72) Inventors: Yanmei Li, Beijing (CN); Liang Li, Beijing (CN)

(73) Assignee: Beijing Gingko Group Biological Technology Co., Ltd., Beijing (CN)

(21) Appl. No.: 13/877,187

(22) PCT Filed: Sep. 30, 2010
(86) PCT No.: PCT/CN10/77542

§ 371 Date: Apr. 1, 2013

(51) Int. Cl. A61K 31/336; A61K 45/06; C07D 303/16

(52) U.S. Cl. CPC A61K 31/336; C07D 303/16; A61K 45/06
USPC 424/752; 549/546; 514/475; 514/78

(57) ABSTRACT

Use of fucoxanthin in the preparation of a product having neuroprotective effect associated with neurodegenerative disorder and improving memory is disclosed in the disclosure. A product having neuroprotective effect associated with neurodegenerative disorder is also disclosed in the disclosure. Fucoxanthin can inhibit oxidative stress of cells and has the effect of preventing or treating Alzheimer's disease and improving memory.

### TECHNICAL FIELD OF THE INVENTION

The disclosure relates to a product having neuroprotective effect associated with neurodegenerative disorder. More particularly, the disclosure relates to the use of fucoxanthin in the preparation of a product for improving memory, and the use of fucoxanthin in the preparation of a product having neuroprotective effect associated with neurodegenerative disorder. The neurodegenerative disorder includes Alzheimer's Disease (AD), Parkinson's disease and Huntington's disease etc.

### BACKGROUND OF THE INVENTION

Natural carotenoids, such as β-carotene, lycopene, lutein and fucoxanthin have been studied extensively because of their anti-cancer properties and remarkable functions of scavenging free radicals. Fucoxanthin, also known as fucoidan, comes from plants including laminaria japonica, gulfweed, wrack, myosoton aquaticum, colpomenia peregrina, chorda filum, wakame, giant kelp, carageen, sargassum miyabei yendo, hijiki, sargassum pallidum, and diatom etc., and is especially abundant in brown algae. The molecular formula of fucoxanthin is C₄₂H₅₈O₆ and its structural formula is as follows:

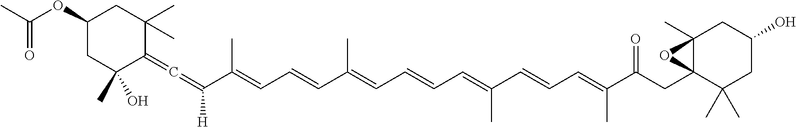

Pure fucoxanthin is a reddish brown crystal, a kind of lutein, a substance to give brown algae a brown color and also a special pigment of brown algae. Fucoxanthin is a potent antioxidant with various biological activities and has the functions to regulate the blood sugar of patients with diabetes effectively, and also may kill various cancer cells (breast cancer, colon cancer and prostatic cancer etc.), thus, Fucoxanthin is of potential values of development and utilization. It is also shown by studies that fucoxanthin has the effect of losing weight.

It is claimed in a literature 2001-335480A published in Japan that fucoxanthin can inhibit rat embryo neuron injuries caused by cerebral ischemia-reperfusion and has protective activity to neurons damaged by ischemia-reperfusion. Cerebral ischemia-reperfusion injury refers to additional damage to brain tissue cells, which is caused by blood perfusion after cerebral ischemia has occurred for a certain period of time. The initiation of the pathological process of ischemic brain damage, which includes primary damage during period of ischemia and secondary damage during period of reperfusion, is ischemia, and cerebral infarction may be caused in worse instances. Currently, inhibition of reperfusion injury is a key part in the treatment of cerebral ischemic stroke. Although the Japanese patent has proven that fucoxanthin can inhibit rat embryo neuron injuries caused by cerebral ischemia-reperfusion and fucoxanthin is claimed to have neuroprotective activity, the neuron injury caused by cerebral ischemia-reperfusion in the Japanese patent refers to nerve damage caused by pathological reasons, and is unrelated with progressive neurodegenerative disorder and age.

The disclosure mainly discusses the use of pure fucoxanthin and the extracts thereof in treating and preventing neurodegenerative disorder. And neurodegenerative disorder which includes AD (dementia) in particular, is a disease with clinical manifestations of deteriorating cognitive and memory functions, progressive loss of activities of daily living, accompanying with various neuropsychiatric symptoms and behavioral disorders. Aging is a major factor of neurodegenerative disorder. Therefore, the disclosure aims to the neuroprotective effect associated with neurodegenerative disorder caused by age and aging.

Neurodegenerative disorder refers to long-term chronic apoptosis of neurons due to genetic factors or environmental factors, including AD (also known as dementia), Parkinson's disease, and Huntington's disease etc. In an era of greying population, neurodegenerative disorder has become one of the major factors that influence the health of the middle-aged and elderly people, and places an enormous economic and social burden to the society, and AD is the most common neurodegenerative ddisease. Epidemiological surveys reveal that AD prevalence is 1% among people over the age of 60 while the prevalence among people at the age of 85 is 30%. It is estimated that the costs for the treatment of AD patients are staggering. The total costs for the treatment of AD patients are 83,900,000,000 dollars per year and on the rise year by year.

The clinical manifestations of AD are deteriorating cognitive and memory functions, progressive loss of activities of daily living, accompanying with various neuropsychiatric symptoms and behavioral disorders. Studies show that one of the major pathological features of AD is senile plaques mainly formed by β-Amyloid (Aβ). Currently, neuron injury caused by Aβdeposition-induced oxidative stress is the major acknowledged pathogenesis of AD. Excessive production, aggregation and deposition of Aβin the brain may cause ion overload in cells to result in imbalance of intracellular environment, promote the production of Reactive Oxygen Species (ROS) and Malondialdehyde (MDA) etc., lead to oxidative stress and lower the level of antioxidant factors including Superoxide Dismutase (SOD), Glutathione Peroxidase (GSH-PX) and the Total Anti-Oxidative Capability (T-AOC) etc. in cells, further causing degeneration and even necrosis of neurons, especially memory-related neurons, and inducing AD. In addition, other hypotheses on the onset of AD include: tau protein abnormalities, heavy metals, vascular factors and virus infection etc. The major brain regions damaged by AD include study and memory-related brain regions, including the cerebral cortex, the basal forebrain and the hippocampus etc.

At present, the Aβ-induced rat cortical neuron damage model has become an important model for researching anti-AD products. The model, which takes primary cultured neurons as samples, has targeting performance of vitro experiments as well as genetic stability of vivo experiments. Therefore the model, which is a powerful tool for screening and developing anti-AD products, is able to prove whether a product has AD resistance and memory-improving effect.

Clinical treatment of AD mainly includes: anti-amyloid protein treatment, neuroprotective therapy, antioxidants, memantine, anti-inflammatory drugs, hormone replacement therapy, and cholinesterase inhibitors etc. However, the therapies above can only temporarily improve the cognitive function and slow down the deterioration thereof to alleviate the symptoms of patients so far, and fail to completely eliminate the cause of illness and cure the disease. Therefore, it is urgent to find a drug to treat AD effectively, and research institutes and top pharmaceutical enterprises all over the world are investing a great deal of human, material and financial resources for this reason.

In recent years, human beings have reached a consensus to “return to the nature”. Therefore, scientists and research companies are interested in finding drugs that effectively treat diseases from natural and ocean plants, and have achieved great progress. It may benefit the treatment of AD by finding effective drugs for AD treatment from natural and ocean plants. In addition, since a lot of therapeutic targets for AD at molecular level have been found, we have reasons to believe that constituents that can delay and treat AD remarkably can be found in natural or ocean plants to further improve memory, and treat and prevent neurodegenerative disorder etc.

During the search for drugs or foods for preventing and treating AD, and improving memory, it is found that fucoxanthin can improve the Aβ-induced rat cortical neuron damage model and inhibit the oxidative stress of the model. It is proven that fucoxanthin has neuroprotective effect associated with neurodegenerative disorder.

### SUMMARY OF THE INVENTION

The disclosure applies primary cortical neurons as a model to discuss the anti-AD activity of fucoxanthin, and the primary cortical neurons closely associating with memory are induced by Aβ active segment Aβ₂₅₋₃₅. The anti-AD activity of fucoxanthin is evaluated by detecting indexes including cell survival rate (MTT), cytomorphology, SOD, GSH-PX, T-AOC and MDA etc. SOD plays an important role in the balance between oxidation and anti-oxidization of organisms. The enzyme is able to remove superoxide anion free radicals and protect cells from damage, and the activity of the enzyme indirectly reflects the ability of organisms to remove ROS. GSH-PX, which is an important enzyme for catalyzing the decomposition of hydrogen peroxide in the body, is able to protect the integrity of cellular membrane structures and functions. The T-AOC can be measured to evaluate the antioxidant capacity of the total anti-oxidant substances in a system. The quantity of MDA reflects the degree of lipid peroxidation in organisms and may indirectly reflect the damage level of cells attacked by free radicals. Hoechst/Propidium Iodide (PI) double staining is applied to cell morphological observation: both PI and Hoechst33342 can bonde with cell nuclear Deoxyribonucleic Acid (DNA) (or Ribonucleic Acid (RNA)). However, PI fails to get through normal cellular membranes while Hoechst is a membrane-permeable fluorescent dye. Therefore, necrotic cells or cells whose cellular membranes are damaged during late apoptosis are stained red by PI while normal cells and cells in early apoptosis or mid-apoptosis are stained blue by Hoechst. However, nucleolus of apoptotic cell present a bright blue color due to concentration. Normal cells (blue), apoptotic cells (bright blue) and necrotic cells (red) can be distinguished according to different colors. However, the accompanying drawings in the specification are colorless, thus the colors are replaced by hollow rounds (blue), grey rounds (bright blue) and black rounds (red).

It is found in researches that, pure fucoxanthin A, fucoxanthin extract powder Fx-powder and oil Fx-oil can improve the survival rate of the rat brain cortical neuron model induced by Aβ₂₅₋₃₅, and protect nerves associated with neurodegenerative disorder, improve the activity of neuron SOD in the culture medium of the rat brain cortical neuron model induced by Aβ₂₅₋₃₅ to a certain degree, reverse the remarkable decrease in the GSH-PX activity caused by neurons damaged by Aβ₂₅₋₃₅, reverse the remarkable decrease in the T-AOC caused by neurons damaged by Aβ₂₅₋₃₅, and significantly reduce the MDA content in the culture medium of neurons damaged by Aβ₂₅₋₃₅. In addition, certain dose dependence is exhibited, and the SOD activity increases with the increase of the drug concentration. It is indicated in the detection result of the aforementioned 4 oxidative damage-related indexes that Fx-powder and Fx-oil have certain oxidative damage resistance which is indicated by the enhancement of enzyme activities associated with peroxide removal and the T-AOC, as well as the reduction of peroxidation damage products in the experiment system. It is indicated in the cell morphological detection result that cell apoptosis and cell necrosis are inhibited by Fx-powder and Fx-oil in different concentrations. Fucoxanthin extracts FX-01 powder and Fx-01 oil have certain neuroprotective effect associated with neurodegenerative disorder and may have the memory-improving activities.

Animal experimental results show that the error response latency of memory acquirement of the animals in fucoxanthin group is prolonged, and the number of errors and the error response rate are reduced during the step-down test and the step-through test. There is a significant difference compared with the blank control group and the results of trainings and repeated tests are consistent. It is indicated that fucoxanthin has a memory improving function. As a healthcare product, fucoxanthin, which plays an important role in health improvement and disease prevention, mainly including improvement of brain development and memory, has a promising future.

The disclosure relates to the novel use of fucoxanthin in improving memory and in the neuroprotective effect associated with neurodegenerative disorder, wherein fucoxanthin can be applied to the neuroprotective effect associated with neurodegenerative disorder.

The use of fucoxanthin according to the disclosure in the preparation of a product having neuroprotective effect associated with neurodegenerative disorder, wherein the product includes a food, a healthcare product and a drug.

The use of fucoxanthin according to the disclosure, wherein the neurodegenerative disorder includes AD, Parkinson's disease, and Huntington's disease.

The use of fucoxanthin according to the disclosure, wherein the product further includes a memory-improving effect. Fucoxanthin is applied to improving memory impairment caused by neurodegenerative disorder, such as memory loss or impairment caused by AD, Parkinson's disease, and Huntington's disease etc.

The use of fucoxanthin according to the disclosure, wherein the dosage form of the fucoxanthin product is selected from at least one kind in a group consisting of powder, an oral solution, a tablet, a capsule, a granule and a pill.

The use of fucoxanthin according to the disclosure, wherein source of fucoxanthin includes a plant source, a microorganism source, or a synthetic compound source.

The use of fucoxanthin according to the disclosure, wherein the plant source of fucoxanthin is seaweed.

The use of fucoxanthin according to the disclosure, wherein the seaweed is selected from a group consisting of laminaria japonica, gulfweed, wrack, myosoton aquaticum, colpomenia peregrina, chorda filum, wakame, giant kelp, carageen, sargassum miyabei yendo, hijiki, sargassum pallidum, and diatom.

The use of fucoxanthin according to the disclosure, wherein the content of fucoxanthin is 0.0001% to 60%, i.e. the content of fucoxanthin may be 0.0001% to 10%, 5% to 15%, 10% to 20% and 15% to 25%, or 25% to 35%, 40% to 50% and 50% to 60%. More preferably, according to the use of the disclosure, the content of fucoxanthin is 0.0001% to 10%.

The use of fucoxanthin according to the disclosure, wherein the content of fucoxanthin in a fucoxanthin extract is 90% to 100%.

The use of fucoxanthin according to the disclosure, wherein the content of fucoxanthin in a fucoxanthin extract is 95% to 100%.

The use of fucoxanthin according to the disclosure, wherein drugs prepared byfucoxanthin include forms of tablets, capsules and pellets.

The use of fucoxanthin according to the disclosure, wherein 0.001 mg to 20 mg of a fucoxanthin active ingredient is taken by a test subject on a daily basis, i.e. 2 mg to 8 mg, 4 mg to 9 mg, 10 mg to 15 mg, or 15 mg to 20 mg of a fucoxanthin active ingredient may be taken by a test subject on a daily basis. The use of fucoxanthin according to the disclosure, wherein 0.001 mg to 10 mg of a fucoxanthin active ingredient is taken by a test subject on a daily basis.

The use of fucoxanthin according to the disclosure, wherein the product contains fucoxanthin with an effective dose to prevent a disease associated with neuro-degeneration.

A product having neuroprotective effect associated with neurodegenerative disorder according to the disclosure, wherein the product contains fucoxanthin.

The product having neuroprotective effect associated with neurodegenerative disorder according to the disclosure, wherein the product includes a food, a healthcare product and a drug.

The product having neuroprotective effect associated with neurodegenerative disorder according to the disclosure, wherein the dosage form of the fucoxanthin product is selected from at least one kind in a group consisting of powder, an oral solution, a tablet, a capsule, a granule and a pill.

The product having neuroprotective effect associated with neurodegenerative disorder according to the disclosure, wherein the source of fucoxanthin includes a plant source, a microorganism source, or a synthetic compound source.

The product having neuroprotective effect associated with neurodegenerative disorder according to the disclosure, wherein the neurodegenerative disorder includes AD, Parkinson's disease, and Huntington's disease.

The product having neuroprotective effect associated with neurodegenerative disorder according to the disclosure, wherein fucoxanthin product further includes a natural extract.

The product having neuroprotective effect associated with neurodegenerative disorder according to the disclosure, wherein the natural extract is selected from a group consisting of a gingko extract, Docosahexaenoic acid (DHA), phosphatidylserine, lecithin, fish oil, Omega-3 and Conjugated Linoleic Acid (CLA).

The product having neuroprotective effect associated with neurodegenerative disorder according to the disclosure, wherein the plant source of fucoxanthin is seedweed.

The product having neuroprotective effect associated with neurodegenerative disorder according to the disclosure, wherein the seaweed is selected from a group consisting of laminaria japonica, gulfweed, wrack, myosoton aquaticum, colpomenia peregrina, chorda filum, wakame, giant kelp, carageen, sargassum miyabei yendo, hijiki, sargassum pallidum, and diatom.

The product having neuroprotective effect associated with neurodegenerative disorder according to the disclosure, wherein the content of fucoxanthin is 0.0001% to 60%, i.e. the content of fucoxanthin may be 0.0001% to 10%, 5% to 15%, 10% to 20% and 15% to 25%, or 25% to 35%, 40% to 50% and 50% to 60%. More preferably, product having neuroprotective effect associated with neurodegenerative disorder according to the disclosure, wherein the content of fucoxanthin is 0.0001% to 10%.

The product having neuroprotective effect associated with neurodegenerative disorder according to the disclosure, wherein the content of fucoxanthin in a fucoxanthin extract is 90% to 100%.

The product having neuroprotective effect associated with neurodegenerative disorder according to the disclosure, wherein the content of fucoxanthin in a fucoxanthin extract is 95% to 100%.

The product having neuroprotective effect associated with neurodegenerative disorder according to the disclosure, wherein 0.001 mg to 20 mg of a fucoxanthin active ingredient is taken by a test subject on a daily basis, i.e. 2 mg to 8 mg, 4 mg to 9 mg, 10 mg to 15 mg, or 15 mg to 20 mg of a fucoxanthin active ingredient may be taken by a test subject on a daily basis.

The product having neuroprotective effect associated with neurodegenerative disorder according to the disclosure, wherein 0.001 mg to 10 mg of a fucoxanthin active ingredient is taken by a test subject on a daily basis.

The use of the product according to the disclosure, the product is applied to improving memory impairment caused by neurodegenerative disorder, such as memory loss or impairment caused by AD, Parkinson's disease, and Huntington's disease etc.

According to the embodiments of the disclosure, the product of the disclosure also has an obvious effect in improving memory. It is indicated in the animal experimental result of an embodiment of the disclosure that the error response latency of memory acquirement of the animals in the fucoxanthin group is prolonged, and the number of errors and the error response rate are reduced during the step-down test and the step-through test. There is a significant difference compared with the blank control group, and it is indicated that fucoxanthin has a remarkable effect in improving memory. Therefore, fucoxanthin may be used for improving memory in a form of pure fucoxanthin, a fucoxanthin extract, and high-content fucoxanthin and a formulation consisting of fucoxanthin in these forms and other raw materials. As a healthcare product, fucoxanthin, which plays an important role in health improvement as well as prevention and treatment of neurological diseases associated with neurodegenerative disorder, mainly including improvement of brain development and memory, can be broadly used.

The use of fucoxanthin of the disclosure in the neuroprotective effect associated with neurodegenerative disorder, especially the AD resistance and memory-improving effect, the novel use of fucoxanthin in the neuroprotective effect associated with neurodegenerative disorder, and a fucoxanthin product having the neuroprotective effect associated with neurodegenerative disorder will be proven through specific examples hereinafter.

### BRIEF DESCRIPTION OF THE DRAWINGS

FIG. 1 shows the morphological detection result of Aβ₂₅₋₃₅ damaged neurons treated with different medicaments. Hollow round: normal cells; grey round: apoptotic cells; black round: necrotic cells.

### DETAILED DESCRIPTION OF THE EMBODIMENTS

The use of fucoxanthin of the disclosure in improving memory will be described hereinafter through specific experiments. Rat brain cortical neurons induced by Aβ-amyloid peptide (Aβ₂₅₋₃₅) are taken as the model, and the neuroprotective effects of a reference substance A (pure fucoxanthin) as well as coarse extracts Fx-01 powder (fucoxanthin powder Fx-powder) and Fx-01 oil (fucoxanthin oil Fx-oil) are detected to evaluate the potential effect of fucoxanthin in resisting cellular damage caused by Aβ₂₅₋₃₅. There are many methods for preparing the pure fucoxanthin A, the fucoxanthin extracts, the fucoxanthin powder and the fucoxanthin oil described hereinafter, and only an example is given for each of them for description.

Preparation method of the pure fucoxanthin A: dissolve a sample containing 1 g of fucoxanthin in a n-hexane solution, pass the solution through a chromatographic column filled with 5 g of silica gel, elute the silica gel column gradually by n-hexane and ethyl acetate in a ratio of 98:2, 95:5, 90:10 and 85:15, monitor thin layer chromatography, spray an alcoholic solution of sulfuric acid for color development, combine flows having the same purity and perform liquid phase detection to obtain the pure fucoxanthin A.

Preparation method of the fucoxanthin powder: see JP2009-261647 and U.S. Ser. No. 12/619,474.

Preparation method of the fucoxanthin oil: add a fucoxanthin extract or pure fucoxanthin to edible oil, and stir uniformly so that fucoxanthin content is within the required range.

### Embodiment 1

### In-Vitro Pharmacological Experiments

### I Experimental Materials and Methods

### 1 Materials

### 1.1 Animals

Zero to four-day old neonatal SD rats purchased from the Department of Laboratory Animal Science of Peking University Health Science Center (PUHSC), laboratory animal license No.: SCXK (Jing) 2006-0025.

### 1.2 Reagents

Aβ₂₅₋₃₅ and MTT, purchased from Sigma Company; T-AOC assay kit, MDA assay kit, GSH-PX assay kit and SOD assay kit, purchased from Nanjing Jiancheng Bioengineering Institute; and cell apoptosis fluorescent Hoechst33342/PI double staining reagent kit purchased from Nanjing KeyGEN Biotech Co., Ltd.

### 2 Method

### 2.1 Primary Culture of Rat Brain Cortical Neurons

1) kill zero to four-day old neonatal rats, immerse the rats in 75% alcohol for 2 to 3 seconds, put the rats in a sterile culture dish, take their brains, and place the brains in a sterile culture dish containing cold D-Hanks liquid, remove meninges and blood vessels, and isolate the cerebral cortex.

2) place the isolated tissues in a sterile culture dish containing a proper amount of 0.125% trypsin solution, cutting up the tissues into a size of about 0.5 mm*0.5 mm*0.5 mm, and place them for 5 minutes at room temperature.

3) transfer the tissue blocks to a 15 ml centrifugal tube, add a proper amount of DMEM-F12 culture medium containing 10% bovine serum to terminate digestion, blow gently with a bend suction pipe to obtain a single cell suspension.

4) filter the cells with a 400-mesh cell strainer.

5) perform centrifugation at 1000 rpm for 5 minutes, discard the supernatant, add a D-Hanks liquid to wash the cells, perform centrifugation at 1000 rpm for 5 minutes and discard the supernatant.

6) add a proper amount of DMEM-F12 culture medium containing 10% bovine serum, count the cells, according to a concentration of 1*10⁵ cells/ml, inoculate cells in a 96-pore plate or a 24-pore plate, which applied with 10 μg/ml of polylysine as a basic layer, place the plate in a CO₂ incubator and culture at 37□ in a CO₂ concentration of 5%.

7) 24 hours later, replace the culture medium with a DMEM-F12 culture medium containing 5 mg/L cytarabine and 10% bovine serum; 24 hours later, replace the culture medium with a DMEM-F12 culture medium containing 2% B-27 and 10% bovine serum; replace the culture medium with a DMEM-F12 culture medium containing 2% B-27 and 10% bovine serum every 2 days subsequently to change the solution.

### 2.2 Experiment Groups and Processing Method

A total of 8 experiment groups are set for cell survival rate detection, including a normal control group, a model group, 5 medicament groups of different concentrations (drug concentrations (the drug concentrations described herein refer to the dry weight of fucoxanthin): 0.39, 0.78, 1.56, 3.12 and 6.25 μg/ml) and a medicament control group (6.25 μg/ml). Other detection indexes: SOD, GSH-PX, T-AOC, MDA and cell morphological observation in which 5 experimental groups are set in total, including a normal control group, a model group and 3 medicament groups of different concentrations (drug concentrations: 0.39, 0.78 and 1.56 μg/ml).

Take cells cultured for 7 days, administer corresponding concentrations of the drug to the medicament groups, add an equal amount of a corresponding solvent to the control group and the model group, incubate for 16 hours, treat groups besides the control group with 5 μM of Aβ₂₅₋₃₅ (pre-aged for 7 days at 37□) for 24 hours, take the cell culture medium to detect the T-AOC, the SOD, the MDA and the GSH-PX subsequently, and measure the cell survival rate by MTT method or perform morphological observation with a Hoechst/PI double staining experiment.

### 2.3 Experimental Index Determination Method

Determination of the cell survival rate by MTT method: culture the cells in a 96-pore plate, administer the drug to protect the cells for 16 hours, after damaging the cells by Aβ₂₅₋₃₅ for 24 hours, add 5 mg/ml of MTT to the culture medium, continue to culture for 4 hours, terminate the culture, remove the culture medium through careful suction, add 200 μl of dimethyl sulfoxide to each pore, measure the absorbance of each pore at Optical Density (OD) 490 nm with an enzyme-labeled instrument after the crystals are completely dissolved and calculate the cell survival rate by taking the control group as 100%.

Hoechst/PI double staining experimental method with reference to the introduction of the kit together with some improvements: add 10 μl of Hoechst 33342 to a 24-pore plate (400 μl culture medium), incubate at 37□ in a dark place for 20 minutes, remove the supernatant through careful suction, wash with PBS for 3 times, add 400 μl Buffer A and 3 μl PI to each pore, incubate at 37□ in a dark place for 15 minutes, remove the supernatant through careful suction, wash with PBS for 3 times, add 400 μl Buffer A to each pore, and observe and photograph with a laser scanning con-focal microscope.

Three experiments of different samples were repeated to detect each index of this experiment (except the Hoechst/PI double staining experiment and the experiment of cell morphological observation), and all results are obtained via the results of three experiments by using statistical analysis.

### II Experimental Results and Discussions

### 1 Influence of the Drug to the Cell Survival Rate

Detect the influence of the reference substance A (A) (pure fucoxanthin) and the coarse extracts thereof FX-01 powder (Fx-powder) and Fx-01 oil (Fx-oil) on the survival rate of the rat brain cortical neuron model induced by Aβ₂₅₋₃₅, and the result is as shown in Table 1. The result indicates that after treating with Aβ₂₅₋₃₅, the cell survival rate decreases remarkably (p<0.01) to 76.03%. It's proven that the Aβ₂₅₋₃₅ treating indeed damages the neurons and the model is successful. However, each drug has certain protective effect on the neurons in the model. Similar to A, the Fx-powder has certain inhibitory effect on neuron damage. However, such effect is not proven to be dose-dependent. The Fx-oil has neuroprotective effect, and is dose-dependent to some extent. The protective effect of the Fx-oil is enhanced with the increase of the drug concentration. By comparing the three drugs, the neuroprotective effect of the reference substance A is greater than that of the Fx-oil, and the neuroprotective effect of the Fx-oil is greater than the Fx-powder with respect to the influence on the cell survival rate. There is no significant difference between the medicament groups and the control group.

TABLE 1
Influence of treating with different drugs on survival rate of neurons damaged by Aβ₂₅₋₃₅

| | Cell survival rate (%) | | | | | | | |
|---|---|---|---|---|---|---|---|---|
| Drug | | | Medication (μg/ml) | | | | | |
| variety | Control | Model | 0.39 | 0.78 | 1.56 | 3.12 | 6.25 | Control (6.25) |
| Fx-powder | 100.00 | 76.03 ± 10.40^{##} | 96.81 ± 14.48* | 96.12 ± 14.51* | 85.25 ± 14.75 | 89.49 ± 16.04 | 90.41 ± 12.74 | 90.42 ± 27.41 |
| Fx-oil | | | 89.49 ± 16.46 | 94.16 ± 14.69 | 99.24 ± 15.50* | 96.40 ± 17.51* | 100.94 ± 11.63** | 102.93 ± 26.96 |
| A (pure | | | 98.45 ± 15.89** | 92.36 ± 17.79 | 98.99 ± 9.52** | 100.98 ± 8.74** | 103.31 ± 11.93** | 104.47 ± 20.70 |
| fuco- | | | | | | | | |
| xanthin) | | | | | | | | |

Note:
compared with the control group,
^{##}p < 0.01;
compared with the model group,
*p < 0.05;
**p < 0.01.

### 2 Influence of Drugs on SOD Activity

The SOD activity can reflect ability of organisms to remove ROS indirectly. The influence of the reference substance A (A) and the coarse extracts thereof FX-01 powder (Fx-powder) and Fx-01 oil (Fx-oil) on the SOD activity in the culture medium of the rat brain cortical neuron model induced by Aβ₂₅₋₃₅ is detected and the result is as shown in Table 2. The result indicates that the SOD activity in the cell culture medium decreases after treating with Aβ₂₅₋₃₅, but there is no significant difference, and each drug can improve the SOD activity of neurons in the model to a certain degree and exhibits certain dose dependence. The SOD activity is improved with the increase of drug concentrations.

TABLE 2
Influence of treating with different drugs on SOD activity in culture
medium of neurons damaged by Aβ₂₅₋₃₅

| | SOD (U/ml) | | | | |
|---|---|---|---|---|---|
| | | | Medication (μg/ml) | | |
| Drug variety | Control | Model | 0.39 | 0.78 | 1.56 |
| Fx-powder | 17.51 ± 0.54 | 14.54 ± 1.56 | 15.49 ± 1.90 | 16.50 ± 1.19 | 17.30 ± 1.31 |
| Fx-oil | | | 15.49 ± 0.97 | 15.45 ± 1.14 | 16.43 ± 2.47 |
| A (pure | | | 15.38 ± 0.71 | 15.72 ± 0.87 | 15.77 ± 0.83 |
| fucoxanthin) | | | | | |

### 3 Influence of Drugs on GSH-PX Activity

Detect the influence of the reference substance A (A) and the coarse extracts thereof FX-01 powder (Fx-powder) and Fx-01 oil (Fx-oil) on the GSH-PX activity in the culture medium of the rat brain cortical neuron model induced by Aβ₂₅₋₃₅, and the result is as shown in Table 3. The result indicates that damage caused by Aβ₂₅₋₃₅ to neurons may result in a remarkable decrease in the GSH-PX activity (p<0.05), while the 3 drugs can improve the GSH-PX activity to a certain degree. However, there is no significant difference compared with the model group and dose dependence is not exhibited.

TABLE 3
Influence of treating with different drugs on GSH-PX activity in culture
medium of neurons damaged by Aβ₂₅₋₃₅

| | GSH-PX (U/ml) | | | | |
|---|---|---|---|---|---|
| | | | Medication (μg/ml) | | |
| Drug variety | Control | Model | 0.39 | 0.78 | 1.56 |
| Fx-powder | 59.22 ± 20.90 | 17.75 ± 17.32^{#} | 39.85 ± 8.43 | 37.00 ± 8.49 | 37.00 ± 5.66 |
| Fx-oil | | | 41.00 ± 14.14 | 54.44 ± 25.14 | 27.22 ± 0.79 |
| A (pure | | | 38.17 ± 6.84 | 61.50 ± 51.62 | 34.00 ± 5.29 |
| fucoxanthin) | | | | | |

Note:
compared with the control group: ^{#}p < 0.05.

### 4 Influence of Drugs on T-AOC

The T-AOC can be measured to evaluate the antioxidant capacity of the total antioxidant substances in the system. The influence of the reference substance A (A) (pure fucoxanthin) and the coarse extracts thereof FX-01 powder (Fx-powder) and Fx-01 oil (Fx-oil) on the T-AOC in the culture medium of the rat brain cortical neuron model induced by Aβ₂₅₋₃₅ is detected, and the result is as shown in Table 4. The result indicates that damage caused by Aβ₂₅₋₃₅ to neurons may result in a remarkable decrease in the T-AOC of the antioxidant substances in the system (p<0.05), while the 3 drugs can improve the T-AOC to a certain degree. The protective effect of the Fx-powder is improved with the increase of the drug concentrations. However, A and the Fx-oil are not dose-dependent although they can improve the T-AOC.

TABLE 4
Influence of treating with different drugs on T-AOC in culture medium of
neurons damaged by Aβ₂₅₋₃₅

| | T-AOC (U/ml) | | | | |
|---|---|---|---|---|---|
| | | | Medication (μg/ml) | | |
| Drug variety | Control | Model | 0.39 | 0.78 | 1.56 |
| Fx-powder | 1.47 ± 0.33 | 0.63 ± 0.05^{#} | 0.78 ± 0.06* | 0.88 ± 0.18 | 1.11 ± 0.06** |
| Fx-oil | | | 1.40 ± 0.12** | 1.48 ± 0.23** | 0.64 ± 0.46 |
| A (pure | | | 1.50 ± 0.54* | 1.26 ± 0.37* | 1.12 ± 0.13** |
| fucoxanthin) | | | | | |

Note:
compared with the control group, ^{#}p < 0.05;
compared with the model group, *p < 0.05, **p < 0.01.

### 5 Influence of Drugs on MDA Content

ROS is generated by organisms through the enzyme system and the non-enzyme system and the ROS is able to attack polyunsaturated fatty acids in biological membranes, and initiate lipid peroxidation to form lipid peroxides, such as MDA. Therefore, the amount of MDA can reflect the degree of lipid peroxidation in organisms, thereby indirectly reflecting the damage degree of cells attacked by free radicals. The influence of the reference substance A (A) (pure fucoxanthin) and the coarse extracts thereof FX-01 powder (Fx-powder) and Fx-01 oil (Fx-oil) on the MDA content in the culture medium of the rat brain cortical neuron model induced by Aβ₂₅₋₃₅ is detected and the result is as shown in Table 5. The result indicates that the damage caused by Aβ₂₅₋₃₅ to the neurons increases the MDA content slightly. The Fx-powder can reduce the MDA content in the culture medium of the damaged neurons remarkably and is dose-dependent, and the protective effect of the Fx-powder increases with the increase of drug concentrations. The Fx-oil also has such effect to protect neurons from oxidative damage and is not evidently dose-dependent. The reference substance A presents certain protective effect at a low dose, but the effect is seemingly reversed at medium and high doses, along with an increase in the MDA content. However, there is no statistical difference compared with the model group.

To sum up, it is indicated in the detection result of the aforementioned 4 oxidative damage-related indexes that the three drugs have certain oxidative damage resistance which is indicated by the enhancement of enzyme activities associated with peroxide removal and the T-AOC in the experimental system, as well as the reduction of peroxidation damage products.

TABLE 5
Influence of treating with different drugs on MDA content in culture
medium of neurons damaged by Aβ₂₅₋₃₅

| | MDA (μmol/ml) | | | | |
|---|---|---|---|---|---|
| | | | Medication (μg/ml) | | |
| Drug variety | Control | Model | 0.39 | 0.78 | 1.56 |
| Fx-powder | 9.54 ± 3.05 | 11.05 ± 3.30 | 3.67 ± 1.62* | 1.72 ± 2.08* | 0.93 ± 0.21** |
| Fx-oil | | | 6.23 ± 2.25 | 0.56 ± 3.47* | 1.36 ± 1.16** |
| A (pure | | | 5.12 ± 1.17* | 14.62 ± 3.82 | 12.34 ± 1.58 |
| fucoxanthin) | | | | | |

Note:
compared with the model group, *p < 0.05; **p < 0.01.

### 6 Neuron Morphological Detection

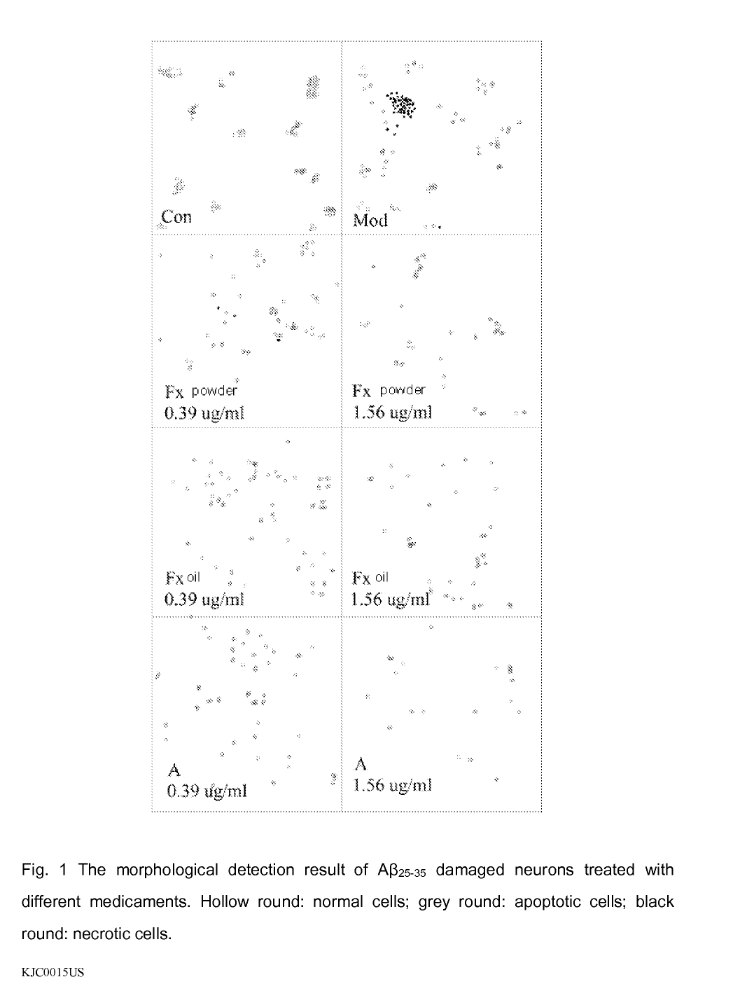

The result of morphological detection is as shown in FIG. 1. The normal control group mainly includes normal cells that are blue, a small amount of bright blue apoptotic cells, and there are no necrotic cells. There is an evident increase in the bright blue apoptotic cells in the model group in which red necrotic cells appear. There is a decrease in the apoptotic cells and the necrotic cells in the Fx-powder 0.39 μg/ml group. The apoptotic cells in the 0.78 μg/ml group decrease and the number of necrotic cells remains the same therein. The necrotic cells decrease, but not in a dose-dependent manner in the 1.56 μg/ml group. The apoptotic cells in the Fx-oil 0.39 μg/ml group and the 0.78 μg/ml group decrease. Both the apoptotic cells and the necrotic cells decrease in the 1.56 μg/ml group. The Fx-oil is dose-dependent to a certain degree. There is a slight decrease in the necrotic cells in the A 0.39 μg/ml group. The apoptotic cells and the necrotic cells decrease in the 0.78 μg/ml group and the 1.56 μg/ml group. A is dose-dependent to a certain degree. By comparing the three drugs, A has the best effect, followed by the Fx-oil and the Fx-powder.

### III Conclusion

1. The reference substance A and the coarse extracts thereof FX-01 powder and Fx-01 oil have certain neuroprotective effect associated with neurodegenerative disorder and may have memory improving activities.

2. By comparing the three drugs, the reference substance A has the best effect, followed by the FX-01 oil and the Fx-01 powder.

3. The three drugs have certain oxidative damage resistance.

### Embodiment 2

### In-Vivo Pharmacological Experiments

### 1 Material and Method

1.1 Material: pure fucoxanthin prepared by our company, and the recommended daily intake for a mouse is equal to the daily dose 4 mg of the human body, male Kuming mice, 6 to 8 weeks old, bodyweight ranging from 18 to 22 g.

1.2 dose and group: take the daily dose, i.e. 4 mg of fucoxanthin of the human body as a dose group, turn the dose into the daily dose of the mice, and set a blank control group.

1.3 Animal laboratory settings: the animal laboratory is Specific Pathogen Free (SPF) in the following conditions: room temperature 22±2□, humidity 60% to 80%.

1.4 Instruments: a step-down instrument and a darkness avoidance instrument.

1.5 Method

1.51 Step-down test: select 20 male mice with a bodyweight of 18 to 22 g, randomly divide the mice into a blank control group and a medication group, administer fucoxanthin to the medication group by lavage, conduct step-down training to the mice after administering the sample to the mice continuously for 30 days, put the animals in a reaction tank for adaption for 3 minutes, introduce a 36V alternating current, and the normal reaction of the animals was to jump back to the insulated platform quickly. Most of the animals might jump onto the copper grids again or jump onto the copper grids for multiple times, and jump back onto the platform after receiving electric shocks. After training once, the animals were placed on the platform in the reaction tank, record the number of electric shocks (number of errors) received by each mouse within 5 minutes as the learning performance, retest 24 hours later, and record the number of animals that received the electric shocks, the latency for the animals to step down the platform for the first time, and number of errors within 3 minutes.

1.52 Step-through test: the selection of animals, the grouping in the test, the doses for the tested objects, the administration methods and the administration time are the same as those in the step-down test, and step-through training was conducted after administering the samples continuously for 30 days. During the experiment, make the mice face back to the entrance of a bright room and put the mice into the bright room, start a timer, the animals passed through the entrance, entered the bright room and received electric shocks, stop the timer, take out the mice and record the time needed for each mouse to be put into the bright room and enter a dark room to receive the electric shocks, i.e. the latency, repeat the test 24 hours later at the same time, and record the latency for each mouse to enter the dark room, the number of errors within 5 minutes, and the number of animals that entered the dark room.

1.6 Data processing: process the obtained data by utilizing an SPSS1010 statistic software package and employ analysis of variance and X² test in the statistic method.

### 2 Results

2.1 Step-down test It can be learned from the following table that during the memory acquisition process (training), the average number of errors of the medication group of the mice orally administered with fucoxanthin for 30 days is less than that of the control group, and there is a significant difference. During the repeated test, the step-down latency of the medication group is longer than that of the control group, the average number of errors of the medication group is less than that of the control group, the error response rate of the medication group is lower than that of the control group, and there are also significant differences (P<0.05).

TABLE 6
Influence of fucoxanthin on memory acquisition of mice (step-down method)

| | | | Latency | | | Error response | |
|---|---|---|---|---|---|---|---|
| | Dose | Number of | Test | Number of errors | | rate (%) | |
| Group | (mg/kg · bw) | animals | (second) | Training | Test | Training | Test |
| Control | 0 | 10 | 89.7 ± 56.4 | 3.1 ± 0.62 | 1.0 ± 0.82 | 100 | 70 |
| group | | | | | | | |
| Medication | 0.42 | 10 | 136.2 ± 25.6* | 1.1 ± 0.56** | 0.3 ± 0.31* | 100 | 50 |
| group | | | | | | | |

Note:
*indicates that p is smaller than 0.05, compared with the blank control group and
**indicates that p is smaller than 0.01.

2.2 Step-through test

It can be learned from the following table that after administering pure fucoxanthin to the mice for 30 days, the average latency of the medication group is longer than that of the control group, the average number of errors of the medication group is less than that of the control group and there is significant difference (P<0.05).

TABLE 7
Influence of fucoxanthin on memory acquisition of mice (step-through method)

| | | | | | | Error |
|---|---|---|---|---|---|---|
| | | | | | Number | response |
| | Dose | Number | Latency (seconds) | | of errors | rate |
| Group | (mg/kg · bw) | of animals | Training | Test | Test | (%) |
| Control | 0 | 10 | 26.5.7 ± 6.4 | 123.41 ± 80.5 | 1.5 ± 0.6 | 100 |
| group | | | | | | |
| Medication | 0.42 | 10 | 40.2 ± 6.5* | 240.4 ± 43.2* | 0.4 ± 0.3** | 50** |
| group | | | | | | |

Note:
*indicates that p is smaller than 0.05, compared with the blank control group and
**indicates that p is smaller than 0.01.

### 3 Discussion

Results of the pharmacological experiments indicate that during the step-down test and the step-through test, the error response latency of the memory acquisition of the animals in the medication group is prolonged, the number of errors, and the error response rate are reduced, and there is a significant difference compared with the control group. It is indicated that fucoxanthin has a memory-improving function. As a healthcare product, fucoxanthin plays an important role in health improvement and disease prevention, including improvement of brain development and memory.

### Embodiment 3

### Materials and Methods

1.1 Materials: Male Kuming laboratory rats, 6 to 8-week old, bodyweight ranging from 18 to 22 g.

1.2 Doses and Groups: follow the formulation of Table 16 and set a blank control group.

1.3 Animal laboratory settings: the animal laboratory is SPF in the following conditions: room temperature 22±2□, humidity 60% to 80%.

1.4 Instruments: a step-down instrument and a darkness avoidance instrument.

1.5 The method is the same as that in the second embodiment

1.7 Data processing: process the obtained data according to the method in the second embodiment.

### 2 Results

2.1 Step-down test

It can be learned from the following table that during the memory acquisition process (training), the average number of errors of the medication group of the mice orally administered with fucoxanthin for 30 days according to the formulation of Table 16 is less than that of the control group, and there is a significant difference. During the repeated test, the step-down latency of the medication group is longer than that of the control group, the average number of errors of the medication group is less than that of the control group, the error response rate of the medication group is lower than that of the control group, and there are also significant differences (P<0.05).

TABLE 8
Influence of the formulation of Table 16 on memory acquisition of mice (step-down method)

| | Administer | | | | | | |
|---|---|---|---|---|---|---|---|
| | a dose of | | | | | | |
| | fucoxanthin | | | | | Error | |
| | effective | Number | Latency | | | response rate | |
| | ingredient | of | Test | Number of errors | | (%) | |
| Group | (mg/kg · bw) | animals | (second) | Training | Test | Training | Test |
| Control | 0 | 10 | 89.7 ± 56.4 | 3.1 ± 0.62 | 1.0 ± 0.82 | 100 | 70 |
| group | | | | | | | |
| Medication | 0.42 | 10 | 145.2 ± 18.6* | 1.1 ± 0.56** | 0.2 ± 0.23* | 100 | 30 |
| group | | | | | | | |

Note:
*indicates that p is smaller than 0.05, compared with the blank control group and
**indicates that p is smaller than 0.01.

3.2 Step-through test

It can be learned from the following table that after orally administering the formulation of Table 16 to the mice for 30 days, the average latency of the medication group is longer than that of the control group, the average number of errors of the medication group is less than that of the control group and there is significant difference (P<0.05).

TABLE 9
Influence of the formulation in Table 14 on memory acquisition of mice (step-through method)

| | Administer a | | | | | |
|---|---|---|---|---|---|---|
| | dose of | | | | | |
| | fucoxanthin | | | | Number | Error |
| | effective | | | | of errors | response |
| | ingredient | Animals | Latency (seconds) | | Tests | rate |
| Group | (mg/kg · bw) | (number) | Training | Test | Test | (%) |
| Control | 0 | 10 | 26.5.7 ± 6.4 | 123.41 ± 80.5 | 1.5 ± 0.6 | 100 |
| group | | | | | | |
| Medication | 0.42 | 10 | 45.1 ± 6.5* | 260.2 ± 33.1* | 0.2 ± 0.1* | 40** |
| group | | | | | | |

Note:
*indicates that p is smaller than 0.05, compared with the blank control group and
**indicates that p is smaller than 0.01.

### 3 Discussion

Results of the pharmacological experiments indicate that during the step-down test and the step-through test, the error response latency of the memory acquisition of the animals in the medication group is prolonged, the number of errors, and the error response rate are reduced, and there is a significant difference compared with the control group. It is indicated that the formulation containing fucoxanthin has a memory-improving function. As a drug and a healthcare product, the formulation plays an important role in health improvement and disease prevention, including improvement of brain development and memory.

### Embodiment 4

### Hard Capsule (Table 8) Prepared from a Fucoxanthin-Containing Extract (Containing 95% of Fucoxanthin)

TABLE 10

| Ingredient | Content |
|---|---|
| Fucoxanthin | 2 mg |
| Ginkgo extract | 98 mg |
| Modified starch | 200 mg |

Dosage for the AD group: 1 to 2 times per day and 1 to 2 capsules each time.

### Embodiment 5

### Film-Coated Tablet (Table 9) Prepared from a Fucoxanthin-Containing Extract (Containing 60% of Fucoxanthin)

TABLE 11

| Ingredient | Content |
|---|---|
| Fucoxanthin | 15 mg |
| Soybean lecithin | 55 mg |
| Rosemary extract | 50 mg |
| Microcrystalline cellulose | 60 mg |
| Starch | 110 mg |
| Sodium carboxymethylcellulose | 2 mg |
| Lemon yellow | 3 mg |
| Titanium dioxide | 5 mg |

Dosage for the AD group: once per day and 1 tablet each time.

### Embodiment 6

### Soft Capsule (Table 10) Prepared from a Fucoxanthin-Containing Extract (Containing 0.1% of Fucoxanthin)

TABLE 12

| Ingredient | Content |
|---|---|
| Fucoxanthin extract | 100 mg |
| Phosphatidylserine | 100 mg |
| Hydrogenated soybean oil | 50 mg |
| Cera flava | 50 mg |
| Soybean oil | 200 mg |

Dosage for the AD group: twice per day and 1 to 2 capsules each time.

### Embodiment 7

### Soft Capsule (Table 11) Prepared from a Fucoxanthin-Containing Extract (Containing 10% of Fucoxanthin)

TABLE 13

| Ingredient | Content |
|---|---|
| Fucoxanthin extract | 50 mg |
| Fish oil | 50 mg |
| Hydrogenated soybean oil | 50 mg |
| Cera flava | 50 mg |
| Soybean oil | 200 mg |

Dosage for the AD group: twice per day and 1 to 2 capsules each time.

### Embodiment 8

### Hard Capsule (Table 12) Prepared from a Fucoxanthin-Containing Extract (Containing 100% of Fucoxanthin)

TABLE 14

| Ingredient | Content |
|---|---|
| Fucoxanthin extract | 4 mg |
| DHA | 20 mg |
| Modified starch | 200 mg |

Dosage for the AD group: 1 to 2 times per day and 1 to 2 capsules each time.

### Embodiment 9

### Soft Capsule (Table 13) Prepared from a Fucoxanthin-Containing Extract (Containing 0.1% of Fucoxanthin)

TABLE 15

| Ingredient | Content |
|---|---|
| Fucoxanthin extract | 50 mg |
| Omega-3 | 50 mg |
| Hydrogenated soybean oil | 50 mg |
| Cera flava | 50 mg |
| Soybean oil | 200 mg |

Dosage for the AD group: twice per day and 1 to 2 capsules each time.

### Embodiment 10

### Soft Capsule (Table 14) Prepared from Oil Containing 1% of Fucoxanthin

TABLE 16

| Ingredient | Content |
|---|---|
| Oil containing 1% of fucoxanthin | 25 mg |
| CLA | 450 mg |
| Soybean oil | 25 mg |

Dosage for the AD group: twice per day and 2 capsules each time.

### Embodiment 11

### Soft Capsule Prepared from an Extract Containing 60% of Fucoxanthin

TABLE 17

| Ingredient | Content |
|---|---|
| Fucoxanthin extract | 5 mg |
| CLA | 90 mg |
| Lecithin | 50 mg |
| Ginkgo extract | 50 mg |
| DHA | 20 mg |
| Phosphatidylserine | 50 mg |
| Soybean oil | 35 mg |

Dosage for the AD group: twice per day and 1 to 2 capsules each time.

### Embodiment 12

### Pellet Prepared from a 100% Fucoxanthin Extract

TABLE 18

| Ingredient | Content |
|---|---|
| Fucoxanthin extract | 20 mg |

### Embodiment 13

### Pellet Prepared from a 90% Fucoxanthin Extract

TABLE 19

| Ingredient | Content |
|---|---|
| Fucoxanthin extract | 15 mg |

It can be understood that those having ordinary skills in the art may make various changes to the details, materials and formulations described here without departing from the principle and scope expressed by the following claims in order to explain the essence of the disclosure.

1. Use of fucoxanthin in the preparation of a product having neuroprotective effect associated with neurodegenerative disorder, wherein the product includes a food, a healthcare product and a drug.

2. The use according to claim 1, wherein the neurodegenerative disorder includes, Parkinson's disease, and Huntington's disease.

3. The use according to claim 2, wherein the product further has a memory-improving effect.

4. The use of fucoxanthin according to claim 1, wherein the source of fucoxanthin includes a plant source, a microorganism source, or a synthetic compound source.

5. The use of fucoxanthin according to claim 4, wherein the plant source of fucoxanthin is seaweed, selected from a group consisting of laminaria japonica, gulfweed, wrack, myosoton aquaticum, colpomenia peregrina, chorda filum, wakame, giant kelp, carageen, sargassum miyabei yendo, hijiki, sargassum pallidum, and diatom.

6. The use of fucoxanthin according to claim 1, wherein the content of fucoxanthin in the product is 0.0001% to 60%; preferably, the content of fucoxanthin in the product is 0.0001% to 10%.

7. (canceled)

8. The use of fucoxanthin according to claim 1, wherein the content of fucoxanthin in a fucoxanthin extract is 90% to 100%; preferably, the content of fucoxanthin in a fucoxanthin extract is 95% to 100%.

9. (canceled)

10. The use of fucoxanthin according to claim 1, wherein 0.001 mg to 20 mg of a fucoxanthin active ingredient is taken by a test subject on a daily basis; preferably, 0.001 mg to 10 mg of a fucoxanthin active ingredient is taken by a test subject on a daily basis.

11. (canceled)

12. The use of fucoxanthin according to claim 1, wherein the product contains fucoxanthin with an effective dose to prevent a disease associated with neuro-degeneration.

13. A product having neuroprotective effect associated with neurodegenerative disorder, wherein the product contains fucoxanthin.

14. The product according to claim 13, wherein the product includes a food, a healthcare product and a drug.

15. The product having neuroprotective effect associated with neurodegenerative disorder according to claim 13, wherein the source of fucoxanthin includes a plant source, a microorganism source, or a synthetic compound source.

16. The product having neuroprotective effect associated with neurodegenerative disorder according to claim 13, wherein the content of fucoxanthin in the product is 0.0001% to 60%; preferably, the content of fucoxanthin in the product is 0.0001% to 10%.

17. (canceled)

18. The product having neuroprotective effect associated with neurodegenerative disorder according to claim 13, wherein the content of fucoxanthin in a fucoxanthin extract is 90% to 100%; preferably, the content of fucoxanthin in a fucoxanthin extract is 95% to 100%.

19. (canceled)

20. The product according to claim 13, wherein the neurodegenerative disorder includes Parkinson's disease, and Huntington's disease.

21. The product according to claim 13, wherein fucoxanthin product further includes a natural extract.

22. The product according to claim 21, wherein the natural extract is selected from a group consisting of a gingko extract, Docosahexaenoic acid (DHA), phosphatidylserine, lecithin, fish oil, Omega-3 and Conjugated Linoleic Acid (CLA).

23. The product according to claim 13, wherein the plant source of fucoxanthin is seaweed, selected from a group consisting of laminaria japonica, gulfweed, wrack, myosoton aquaticum, colpomenia peregrina, chorda filum, wakame, giant kelp, carageen, sargassum miyabei yendo, hijiki, sargassum pallidum, and diatom.

24. The product according to claim 13, wherein 0.001 mg to 10 mg of a fucoxanthin active ingredient is taken by a test subject on a daily basis; preferably, 0.001 mg to 10 mg of a fucoxanthin active ingredient is taken by a test subject on a daily basis.

25. (canceled)

26. Use of the product according to claim 13 in improving memory.
